(19) United States
(12) Patent Application Publication
(10) Pub. No.: US 2012/0027737 A1
(43) Pub. Date: Feb. 2, 2012

(54) USE OF PROBIOTICS TO AMELIORATE DIET-INDUCED INSULIN RESISTANCE

(75) Inventors: Thomas Dyrmann Leser, Frederiksberg (DK); Thomas Gunnarsson, Malmoe (SE); Jens Kildsgaard, Holte (DK); Janni Wandahl Pedersen, Alleroed (DK); Bénédicte Flambard, Frederiksberg (DK)

(73) Assignee: CHR-HANSEN A/S

(21) Appl. No.: 13/256,852

(22) PCT Filed: Mar. 19, 2010
(86) PCT No.: PCT/EP2010/053618

§ 371 Date: Sep. 15, 2011

(30) Foreign Application Priority Data
Mar. 25, 2009 (EP) 09156121.7

(51) Int. Cl. A61K 35/74; A61P 1/14; A61P 1/00; C12N 1/20; A61P 3/04

(52) U.S. Cl. 424/93.45; 435/252.9; 424/780

(57) ABSTRACT

The invention relates to the use of a composition comprising probiotic bacteria that regulate expression of key components involved in diet-induced insulin resistance for ameliorating or preventing diet-induced insulin resistance. The use of the probiotic strain and/or a fraction of said strain and/or metabolite of said strain for the manufacture of a medicament or a food or feed product to ameliorate diet-induced insulin resistance and help to obtain optimal body weight of a mammal is disclosed. Preferably, the composition comprises at least one probiotic Lactobacillus acidophilus strain and/or a fraction of said strain and/or metabolite of said strain for ameliorating or preventing diet-induced insulin resistance, said composition characterized by up-regulating expression of the ANGPTL4 gene encoding for FIAF in the intestine, down-regulating expression of the Elovl6 gene in the intestine as well as down-regulating expression of the SCD1 gene in skeletal muscles of a mammal, and wherein the probiotic strain is selected from the group of strains consisting of Lactobacillus acidophilus strain LA-5 (DSM13241).

### FIELD OF THE INVENTION

The invention relates to the use of a composition comprising probiotic bacteria that regulate expression of key components involved in diet-induced insulin resistance. Consumption of the probiotic strain may ameliorate diet-induced insulin resistance and help to obtain optimal body weight of a mammal.

### BACKGROUND OF THE INVENTION

### Diet-Induced Insulin Resistance

In a person with normal metabolism, insulin is released from the beta cells of the Islets of Langerhans located in the pancreas after eating, and it signals insulin-sensitive tissues in the body (e.g., muscle and adipose tissue) to absorb glucose. This lowers blood glucose levels. The beta cells reduce their insulin output as blood glucose levels fall, with the result that blood glucose is maintained at approximately 5 mmol/L (mM) (90 mg/dL). In an insulin-resistant person, normal levels of insulin do not have the same effect on muscle and adipose cells, with the result that glucose levels stay higher than normal. To compensate for this, the pancreas in an insulin-resistant individual is stimulated to release more insulin. The most common type of insulin resistance is associated with a collection of symptoms known as metabolic syndrome (insulin resistance, high blood pressure; central obesity, decreased HDL cholesterol; elevated triglycerides) and prediabetes (www.wikipedia.org).

According to the CDC (Centers for Disease Control and Prevention), prediabetes raises the risk of developing type 2 diabetes, heart disease, stroke, and eye disease. About 54 million individuals in the United States aged 21 years and older have prediabetes, 12 million of who are overweight and between the ages of 45-74. In the United States, approximately one of every three persons born in 2000 will develop diabetes in his or her lifetime. The lifetime risk of developing diabetes is even greater for ethnic minorities: two of every five African Americans and Hispanics, and one of two Hispanic females, will develop the type 2 diabetes.

A number of epidemiological as well as experimental studies have revealed that a lifestyle which involves excess caloric intake, in particular excess intake of carbohydrate and saturated fatty acids, is the principal cause to lifestyle-related insulin resistance¹. Insulin resistance is a strongly associated with obesity and is the major pathogenic indicator of obesity-related diseases such as metabolic syndrome, hypertension, cardiovascular pathology, and non-alcoholic fatty liver disease^{2,3}.

While the precise molecular cause to lifestyle-related insulin resistance remains obscure, evidence suggest that accumulation of lipids activate pro-inflammatory and stress-responsive signals which in turn result in insulin-resistance through abnormal phosphorylation or degradation of the insulin signaling molecules⁴⁻⁷.

Studies have shown that the endogenous fatty acid synthesis pathway is crucial for energy metabolism and insulin sensitivity. Lipogenic enzymes belonging to this pathway, e.g. stearoyl-CoA desaturase 1 (SCD-1) and Elovl6, which elongates long-chain saturated and unsaturated fatty acids, as well as certain factors involved in the catabolism of lipids, e.g. the fasting-induced adipocyte factor (FIAF), have been shown to modulate insulin sensitivity in the liver⁸⁻¹⁰.

### Body Weight Management

The healthy, well functioning body of a mammal (including humans) is characterized by an optimal weight. The specific optimal weight varies widely according to species, gender, age, type of body stature, level of physical activity etc. of the individual mammal. It is however clear that an optimal body weight range can be established for any individual mammal, and that extensive over- as well as under-weight have drastic negative effects on the health and wellbeing of the individual.

The maintenance of the optimal body weight is complex and multifactorial (NIH 1998). It involves a multitude of signaling pathways and metabolic processes as well as a spectrum of genetic and environmental factors. Present clinical evidence indicates that a multi-faceted intervention involving several signaling pathways and metabolic processes is required to obtain an effect full treatment of obesity.

Within the last decade it has become increasingly clear that the healthy mammalian body also has developed a number of intricate mechanisms that regulate the feed intake during periods of surplus by regulating our satiety. Many of these mechanisms seem to involve specific responses to certain components in the food or the gastrointestinal microbiota, and the molecular details of more signaling pathways of lipid metabolism have been revealed and have shown to involve specific molecules/hormones. Depending on their specific levels (presence or absence) such specific signaling molecules/hormones may influence fatty acid metabolism. FIAF is an example of a molecule involved in the regulation of fatty acid metabolism and associated with insulin resistance.

### The Biology and Physiology of FIAF.

FIAF (also known as fasting-induced adipocyte factor or angiopoietin-like protein 4 [ANGPTL4]), Ensembl: ENSG00000167772, is encoded by the human chromosome 19 band p13.3. This gene is a member of the angiopoietin/angiopoietin-like gene family and encodes a glycosylated, secreted protein with a fibrinogen C-terminal domain. Alternatively spliced transcript variants encoding different isoforms have been described. The gene is induced under hypoxic conditions in endothelial cells and is the target of peroxisome proliferation activators. The encoded protein is a serum hormone directly involved in regulating glucose homeostasis, lipid metabolism, and insulin sensitivity and also acts as an apoptosis survival factor for vascular endothelial cells. The encoded protein may play a role in several cancers and it has been shown to prevent the metastatic process by inhibiting vascular activity as well as tumor cell motility and invasiveness. Furthermore, decreased expression of this protein has been associated with type 2 diabetes¹¹⁻¹⁵.

Animal studies have shown that FIAF expression is regulated by the gastrointestinal microbiota. For example, germ-free mice (GF) contain significantly less body fat compared to conventional mice with a normal microbiota (CONV) in spite of higher daily chow consumption² (see FIG. 1). Increased body fat content in CONV mice is due to suppression of intestinal gene expression of FIAF by the microbiota. Conventionalization of adult GF mice causes a 50% reduction in gut epithelial FIAF expression. FIAF is an inhibitor of lipoprotein lipase (LPL). LPL encodes lipoprotein lipase, which is expressed in heart, muscle, and adipose tissue. LPL has the dual functions of triglyceride hydrolase and ligand/bridging factor for receptor-mediated lipoprotein uptake. LPL is, thus, a key regulator of fatty acid release from triglyceride-rich lipoproteins in muscle, heart, and fat. Increased adipocyte LPL activity leads to increased cellular uptake of fatty acids and adipocyte triglyceride accumulation according to the model presented in FIG. 2.

The elongation of long-chain fatty acids (ELOVL) family member 6 (Elovl6, also known as LCE and FACE). is another example of a molecule involved in the regulation of fatty acid metabolism and is clearly associated to insulin resistance.

### The Biology and Physiology of ELOVL6

ELOVL6, gene ID: ENSG00000170522, is encoded by the human chromosome 4 band q25. ELOVL6 encodes the elongase (EC 6.2.1.3) that catalyzes the conversion of palmitate to stearate.

Mice with a targeted disruption in the gene for Elovl6 (Elovl6−/−) are resistant to diet-induced insulin resistance. This is observed despite hepatosteatosis and obesity being similar to that of their wild-type litter mates. Protection against diet-induced insulin resistance in Elovl6−/− mice is partially due to restoration of hepatic insulin receptor substrate-2 and suppression of hepatic protein kinase C ε, resulting in restoration of Akt phosphorylation¹⁶.

It has been suggested that inhibition of this elongase could be a new therapeutic approach for the treatment of insulin resistance, diabetes, cardiovascular disease, and other metabolic diseases¹⁷.

Interestingly, the elongation of very long-chain fatty acids in addition to ELOVL6 also involves another key enzyme, stearoyl-CoA desaturase (SCD, also known as SCD1; EC 1.14.19.1).

### The Biology and Physiology of Stearoyl-CoA Desaturase-1 (SCD1)

Stearoyl-CoA desaturase (SCD; EC 1.14.19.1) is an iron-containing enzyme that catalyzes a rate-limiting step in the synthesis of unsaturated fatty acids. SCD-1 is encoded by a gene on human chromosome 10q24.31 with gene ID: Ensembl:ENSG00000099194.

Stearoyl-CoA desaturase-1 (SCD1) determines fatty acid partitioning into lipogenesis or fatty acid β-oxidation in muscle tissue. Up-regulation of SCD1 is seen in obese individuals and results in accumulation of intramyocellular triacylglycerol (IMTG). Human obesity is associated with abnormal accumulation of neutral lipids within skeletal myofibers. This phenomenon occurs in concert with reduced insulin stimulated glucose transport and impaired insulin signal transduction. Pharmacological and genetic manipulations that deplete IMTG restore insulin sensitivity. Hulver et al. (2005)¹⁸ have identified a linear relationship between Body Mass Index (BMI) and the expression of SCD in muscles in humans. In vitro studies have shown that over-expression of SCD1 in myotubes from lean subjects altered fatty acid partitioning in a manner that resembled the high rates of muscle triacylglycerol (TAG) synthesis and low rates of fatty acid oxidation observed with obesity. The authors proposed “that elevated expression of SCD1 in skeletal muscle may represent a mechanism contributing to reduced fatty acid oxidation, increased IMTG synthesis and progression of the metabolic syndrome”, and further, “that pharmacological targeting of muscle SCD1 and/or its upstream regulators could provide new opportunities for preventing and/or treating obesity and its related co-morbidities¹⁸.

### Probiotics

Probiotic microorganisms have been defined as “Live microorganisms which when administered in adequate amounts confer a health benefit on the host” (FAO/WHO 2002).

It has been described that certain probiotic bacterial strains may have the ability to modulate the expression of some of the genes involved in lipid metabolism and insulin resistance.

WO 2008 083157 A2 describes a method for modulating body fat and/or weight loss which comprise altering the amount of or the activity of a FIAF and, at the same time, the amount of or the activity of an AMPK polypeptide in the subject. The method may involve certain probiotics.

US 2008/0019911 A1 describes a method for Increasing insulin sensitivity by administering angiopoietin like protein-4 (ANGPTL4=FIAF) polypeptide to a patient.

Bäckhed et al. (2004) describes that gut microbiota is an environmental factor that increases fat storage, presumably through down-regulation of FIAF^{2,19}.

EP1456351B describes a pure strain of Streptococcus thermophilus ssp. salivarius (CD8, DSM14667) and its use for prevention/treatment of insulin resistance or obesity.

WO07043933A describes that certain probiotics, preferably Lactobacillus casei F19 (LMG P-17806), Lactobacillus acidophilus NCFB 1748, and Bifidobacterium lactis Bb12 can be used simultaneously (!) for controlling weight gain, preventing obesity, increasing satiety, prolonging satiation, reducing food intake, reducing fat deposition, improving energy metabolism, enhancing insulin sensitivity, treating obesity and treating insulin insensitivity.

Current literature indicates that the mechanism behind development of insulin resistance is complex and multifactorial. It seems to involve more signaling pathways and metabolic processes as well as a spectrum of genetic and environmental factors. It seems plausible that a multi-faceted intervention involving more such signaling pathways and/or metabolic processes is required to obtain an efficient treatment of the disease.

However to the best of our knowledge there is no report of a probiotic bacterium that is able to modulate several of the dominators involved in diet-induced insulin resistance.

### SUMMARY OF THE INVENTION

The invention relates to the use of probiotic Lactobacillius acidophilus and/or a fraction and/or metabolite of said strain to for ameliorating or preventing diet-induced insulin resistance in a mammal. To the surprise of the inventors, compositions comprising probiotic Lactobacillus acidophilus strain LA-5 (DSM13241) are able to up-regulate the expression of the ANGPTL4 gene encoding for FIAF in the intestine, and also to down-regulate expression of the Elovl6 gene in the intestine and down-regulate expression of the SCD1 gene in skeletal muscles of a mammal. ANGPTL4, Elovl6 as well SCD1 codes for enzymes that are strongly associated with the development of diet-induced insulin resistance, and that data make it highly plausible that the coordinately increased expression of the ANGPTL4 gene and the reduced expression of both the Elovl6 and the SCD1 genes induced by LA-5 will ameliorate, prevent or even treat the disease.

A further aspect of the invention is the use of a composition comprising the LA-5 strain and/or a fraction and/or metabolite of said strain according to the invention for the preparation of a composition for body weight management of a mammal.

One particularly interesting aspect is the use of a composition comprising the strain and/or a fraction and/or metabolite of said strain according to the invention for the preparation of a medicament for the treatment of overweight or obesity. It is well known that obesity (BMI≧30) but also overweight (i.e. BMI 25-30) may have serious medical implications and that obese as well as overweight individuals may benefit from a weight reduction. However, even normal or near-normal weight individuals (i.e. BMI 18.5-24.9) who do not suffer under medical implications due to overweight may find it attractive to maintain or strive for an optimal body weight for cosmetic reasons. Thus, one additional aspect of the invention is the use of a composition comprising the strain and/or a fraction and/or metabolite of said strain according to the invention in a cosmetic method for reducing body weight in a non-obese, non-overweight subject having a Body Mass Index (BMI) less than 25, said method comprises providing a composition comprising at least one strain of Bifidobacterium animalis subsp. lactis and/or Lactobacillus acidophilus and/or a fraction of said strain and/or metabolite of said strain, wherein said composition is characterized by up-regulating expression of the ANGPTL4 gene encoding for FIAF in the intestine, down-regulating expression of the Elovl6 gene in the intestine as well as down-regulating expression of the SCD1 gene in skeletal muscles of a mammal.

The composition comprising the strain and/or a fraction and/or metabolite of said strain according to the invention may be formulated in both liquid and solid dosage forms. In the latter case, the product may be powdered and formed into tablets, granules or capsules or simply mixed with other food ingredients to form a functional food. Accordingly in one aspect the composition comprising the strain and/or a fraction and/or metabolite of said strain according to the invention is used for the preparation of a food or feed intended to ameliorate or prevent diet-induced insulin resistance of a mammal.

### DEFINITIONS

Prior to a discussion of the detailed embodiment of the invention a definition of specific terms related to the main aspects of the invention is provided.

By the expression “diet-induced insulin resistance” is referred to a condition in which normal amounts of insulin are inadequate to produce a normal insulin response from fat, muscle and liver cells. Insulin resistance has also been arbitrarily defined as the requirement of 200 or more units of insulin per day to attain glycemic control and to prevent ketosis. “Diet-induced” indicates that the condition is induced by a diet high in saturated fat and carbohydrates.

The syndromes of insulin resistance actually make up a broad clinical spectrum, which includes obesity, glucose intolerance, diabetes, and the metabolic syndrome, as well as an extreme insulin-resistant state. Many of these disorders are associated with various endocrine, metabolic, and genetic conditions. These syndromes may also be associated with immunological diseases and may exhibit distinct phenotypic characteristics.

By the expression “risk factors involved in overweight and/or obesity” is referred to one or more the many biochemical factors that are negatively involved in the development of overweight and/obesity. One particularly interesting group of such risk factors is the so-called FIAF molecule, polypeptide or hormone. By the term “FIAF” is referred to the hormone also known as “fasting-induced adipocyte factor” or “angiopoietin-like protein 4” which is a serum hormone directly involved in regulating glucose homeostasis, lipid metabolism, and insulin sensitivity and also acts as an apoptosis survival factor for vascular endothelial cells. The encoded hormone may play a role in several cancers and it also has been shown to prevent the metastatic process by inhibiting vascular activity as well as tumor cell motility and invasiveness. Decreased expression of this protein has been associated with type 2 diabetes and weight gain in mice.

As used herein the term “BMI” designates body mass index. BMI is a measure of the weight of a person scaled according to height. It is defined as the individual's body weight divided by the square of their height (weight measured in kilograms, height in meters). The formula universally used in medicine produces a unit of measure of kg/m². According to the US Department of Health & Human Services a BMI below 18.5 indicates underweight, 18.5-24.9 normal weight, 25-29.9 overweight and a BMI of 30 and above indicates obesity. It should be noted that not only obesity but also overweight (BMI 25-29.9) increases the risk of mortality in adults²⁰. Accordingly overweight is not only of relevance because of cosmetic indications but also for its medical implications.

By the expression “probiotics or probioticum” is referred to a composition which comprises probiotic microorganisms. Probiotic bacteria are defined as microbial cells that have a beneficial effect on the health and well-being of the host. Probiotic microorganisms have been defined as “Live microorganisms which when administered in adequate amounts confer a health benefit on the host” (FAO/WHO 2002).

By the expression “prebiotic” is referred to a composition or a component of a composition which increases the number of probiotic bacteria in the intestine. Thus, prebiotics refer to any non-viable food component that is specifically fermented in the colon by indigenous bacteria thought to be of positive value, e.g. bifidobacteria and lactobacilli. The combined administration of a probiotic strain with one or more prebiotic compounds may enhance the growth of the administered probiotic in vivo resulting in a more pronounced health benefit, and is termed synbiotic.

Embodiments of the present invention are described below, by way of examples only.

### DETAILED DISCLOSURE OF THE INVENTION

The invention relates to the use of the probiotic Lactobacillius acidophilus strain LA-5 and mutations and variations thereof to modify key components in the fatty acid metabolism that are associated with the onset of diet-induced insulin resistance in mammals. To the surprise of the inventors, compositions comprising certain live probiotic Lactobacillus acidophilus LA-5 bacteria are able specifically to enforce the expression of three genes, the ANGPTL4 gene, the Elovl6 gene and the SCD1 gene in a way that makes it highly plausible that Lactobacillus acidophilus LA-5 can ameliorate, prevent or even treat diet-induced insulin resistance and diseases related thereto.

The strain Lactobacillus acidophilus strain LA-5 (DSM13241) was deposited according to the Budapest Treaty on the International Recognition of the Deposit of Microorganisms for the Purposes of Patent Procedure at DSMZ (Deutsche Sammlung von Mikroorganismen and Zellkulturen GmbH) on Sep. 30, 2003 under accession number DSM13241. The LA-5 strain is commercially available from Chr. Hansen NS, 10-12 Boege Alle, DK-2970 Hoersholm, Denmark.

As illustrated in example 1, the probiotic Lactobacillus acidophilus LA-5 (DSM13241) is particularly effective in decreasing the expression of the ELOVL6 gene. In this experiment the piglets were treated for a two-week treatment period. Then the piglets were killed and tissues were sampled. The samples were subjected to Q-PCR analysis of gene expression as described in the example.

As further illustrated in example 2, Lactobacillus acidophilus LA-5 (DSM13241) is also particularly effective in down-regulating expression of the ELOVL6 elongase and the stearoyl-CoA desaturase (SCD, also known as SCD1; EC 1.14.19.1). Both are key enzymes involved in the biosynthesis of monounsaturated fatty acids (Samulin 2009).

Finally, example 3 describes that Lactobacillus acidophilus LA-5 bacteria are able to induce the expression of the ANGPTL4 (=FIAF) gene particularly in the distal part of the small intestine and in the colon of a mammal.

It is contemplated that strains directly derived from Lactobacillus acidophilus LA-5 (DSM13241) are likely to retain their probiotic features. Accordingly, one preferred embodiment of the invention is the use of a mutant strain of Lactobacillus acidophilus strain LA-5 (DSM13241), wherein the mutant strain is obtained by using DSM13241, and wherein the mutant has retained or further improved the ability to up-regulate expression of the ANGPTL4 gene or/and further improved the ability to down-regulate expression of the Elovl6 gene in the intestine or/and further improved the ability to down-regulate expression of SCD1 gene in skeletal muscles of said mammal.

These data indicate that oral intake of Lactobacillus acidophilus LA-5 will reduce adipocyte accumulation of triglycerides through their up-regulation of intestinal ANGPTL4 expression according to the model shown in FIG. 3. Furthermore, a decrease in Stearoyl-CoA desaturase-1 (SCD1) gene expression seems to result in an increased fatty acid oxidation and to be inversely related to the progression of the metabolic syndrome. When one adds recent reports implicating increased levels of ELOVL6 in lipogenesis adipocyte development²¹ it seems clear that up-regulation of ANGPTL4 expression and down-regulation of SCD1 and ELOVL6 expression represent a productive strategy for reducing lipid accumulation in tissues with the aim of treating or preventing obesity and obesity-related disorders, in addition to diet-induced insulin resistance.

Without wishing to be bound by theory it is perceivable that not only living Lactobacillus acidophilus bacteria but also a fraction of said bacteria or even a metabolite of said strain can be used for the preparation of a composition for administration to a mammal for modulating expression of the Elovl6, the SCD1 and the ANGPTL4 gene in the animal.

Obesity is a major risk factor for developing a number of diseases and symptoms. According to The Endocrine Society or The Hormone Foundation (http://www.obesityinamerica.org) overweight and obese people are at an increased risk for developing the following conditions: Cardiovascular diseases (e.g. atherosclerosis, hypertension, stroke, congestive heart failure, Angina pectoris), type 2 diabetes mellitus, obesity-related hypoventilation, back and joint problems, non-alcoholic fatty liver disease, gastroesophageal reflux disease, reduced fertility, hypothyroidism, dyslipidemia, hyperinsulinemia, cholecystitis, cholelithiasis, osteoarthritis, gout, sleep apnea and other respiratory problems, polycystic ovary syndrome (PCOS), pregnancy complications, psychological disorders, uric acid nephrolithiasis (kidney stones), stress urinary incontinence and increased incidence of certain cancers (e.g. cancer of the kidney, endometrium, breast, colon and rectum, esophagus, prostate and gall bladder).

Accordingly, yet an embodiment of the invention is the use of Lactobacillus acidophilus LA-5 and/or a mutant of LA-5 and/or a fraction and/or a metabolite of said strains of for the preparation of a composition or medicament for the prevention and/or treatment of anyone of the above mentioned diseases or conditions.

Many probiotics are used for the manufacture of food or feed products; consequently a further important aspect of the invention is the provision of a human or animal food or feed composition comprising the Lactobacillus acidophilus strain LA-5 (DSM13241) and/or a fraction and/or metabolite of said strain to control or stabilize the weight gain of a mammal. Such food or feeds are frequently referred to as functional food or feed.

When preparing such food or feed products manufacturers usually make use of so-called starter cultures being cultures used to process food and feed products. Starter cultures are widely used in the diary industry. Typically, starter cultures impart specific features to various food or feed products. It is a well established fact that the consistency, texture, body and mouth feel is strongly related to the EPS production of the starter culture used to prepare the food or feed.

The present invention also devices a method of manufacturing a food or feed product comprising adding a starter culture composition comprising Lactobacillus acidophilus strain LA-5 (DSM13241) or a mutant strains thereof to a food or feed product starting material and keeping the thus inoculated starting material under conditions where the lactic acid bacterium is metabolically active, and thereby to obtain a food or feed product to control or stabilize the weight gain of a mammal.

By the expression “prebiotic” is referred to a composition or a component of a composition which increases the number of probiotic bacteria in the intestine. Thus, prebiotics refer to any non-viable food component that is specifically fermented in the colon by indigenous bacteria thought to be of positive value, e.g. bifidobacteria and lactobacilli. The combined administration of the probiotic LA-5 strain with one or more prebiotic compounds may enhance the growth of the administered probiotic in vivo resulting in a more pronounced health benefit. Therefore one further embodiment of the invention is the use of a composition comprising living probiotic bacteria according to the invention in combination with at least one prebiotic. An embodiment wherein the prebiotic is selected from the group: inulin, a transgalacto-oligosaccharide, palantinoseoligosaccharide, soybean oligosaccharide, gentiooligosaccharide, oxylooligomers, nondegradable starch, lactosaccharose; lactulose, lactitol, maltitol, FOS (fructo-oligosaccharides), GOS (galacto-oligosaccharides) and polydextrose, is especially preferred.

### The Invention Presented in the Form of Claims

Preferred aspects and embodiments of the invention may be presented in the form of so-called claims. These are given below.

1. A composition comprising at least one probiotic Lactobacillius acidophilus strain and/or a fraction of said strain and/or metabolite of said strain for ameliorating or preventing diet-induced insulin resistance, said composition is characterized by up-regulating expression of the ANGPTL4 gene encoding for FIAF in the intestine, down-regulating expression of the Elovl6 gene in the intestine as well as down-regulating expression of the SCD1 gene in skeletal muscles of a mammal.

2. The composition according to claim 1, wherein the strain is selected from the group of strains consisting of Lactobacillus acidophilus strain LA-5 (DSM13241), and a mutant strain thereof, wherein the mutant strain is obtained by using DSM13241, and wherein the mutant has retained or further improved the ability to up-regulate expression of the ANGPTL4 gene or/and further improved the ability to down-regulate expression of the Elovl6 gene in the intestine or/and further improved the ability to down-regulate expression of SCD1 gene in skeletal muscles of said mammal.

3. The composition according to any of the preceding claims for ameliorating, treating or preventing a disease or condition selected from the group of obesity and obesity-related diseases consisting of obesity-induced insulin resistance, cardiovascular diseases (e.g. atherosclerosis, hypertension, stroke, congestive heart failure, Angina pectoris), type 1 diabetes mellitus, type 2 diabetes mellitus, metabolic syndrome, leptin resistance, obesity-related hypoventilation, back and joint problems, non-alcoholic fatty liver disease, gastroesophageal reflux disease, reduced fertility, hypothyroidism, dyslipidemia, hyperinsulinemia, cholecystitis, cholelithiasis, osteoarthritis, gout, sleep apnea and other respiratory problems, polycystic ovary syndrome (PCOS), pregnancy complications, psychological disorders, uric acid nephrolithiasis (kidney stones), stress urinary incontinence and certain cancers (e.g. cancer of the kidney, endometrium, breast, colon and rectum, esophagus, prostate and gall bladder).

4. A cosmetic method for reducing body weight in a non-obese, non-overweight subject having a Body Mass Index (BMI) less than 25, said method comprise providing a composition comprising at least one strain of a probiotic bacterial strain and/or a fraction of said strain and/or metabolite of said strain, wherein said composition is characterized by up-regulating expression of the ANGPTL4 gene encoding for FIAF in the intestine, down-regulating expression of the Elovl6 gene in the intestine as well as down-regulating expression of the SCD1 gene in skeletal muscles of a mammal.

5. A cosmetic method for reducing body weight in a non-obese subject, said method comprise providing a composition comprising at least one strain of a probiotic bacterial strain and/or a fraction of said strain and/or metabolite of said strain, wherein said composition is characterized by up-regulating expression of the ANGPTL4 gene encoding for FIAF in the intestine, down-regulating expression of the Elovl6 gene in the intestine as well as down-regulating expression of the SCD1 gene in skeletal muscles of a mammal.

6. The cosmetic method according to any of claim 4 or 5, wherein the strain is selected from the group of strains consisting of Lactobacillus acidophilus strain LA-5 (DSM13241), and a mutant strain thereof, wherein the mutant strain is obtained by using DSM13241, and wherein the mutant has retained or further improved the ability to up-regulate expression of the ANGPTL4 gene or/and further improved the ability to down-regulate expression of the Elovl6 gene in the intestine or/and further improved the ability to down-regulate expression of SCD1 gene in skeletal muscles of said mammal.

7. The composition according to any of the preceding claims, wherein the at least one strain and/or a fraction and/or metabolite is used for the preparation of a food or feed intended to ameliorate or prevent diet-induced insulin resistance of a mammal.

8. The composition according to any of the preceding claims, wherein the at least one strain and/or a fraction and/or metabolite is combined with at least one prebiotic.

9. A composition according to claim 17, wherein the at least one strain and/or a fraction and/or metabolite is combined with at least one prebiotic, wherein the at least one prebiotic is selected from the group consisting of: inulin, a transgalacto-oligosaccharide, palantinoseoligosaccharide, soybean oligosaccharide, gentiooligosaccharide, oxylooligomers, nondegradable starch, lactosaccharose; lactulose, lactitol, maltitol, FOS (fructo-oligosaccharides), GOS (galacto-oligosaccharides), and polydextrose.

10. A use of at least one strain one probiotic bacterial strain and/or a fraction of said strain and/or metabolite of said strain for the preparation of a medicament for administration to a mammal for treating, ameliorating or preventing diet-induced insulin resistance, said composition is characterized by up-regulating expression of the ANGPTL4 gene encoding for FIAF in the intestine, down-regulating expression of the Elovl6 gene in the intestine as well as down-regulating expression of the SCD1 gene in skeletal muscles of a mammal.

11. The use according to claim 10, wherein the strain is selected from the group of strains consisting of Lactobacillus acidophilus strain LA-5 (DSM13241), and a mutant strain thereof, wherein the mutant strain is obtained by using DSM13241, and wherein the mutant has retained or further improved the ability to up-regulate expression of the ANGPTL4 gene or/and further improved the ability to down-regulate expression of the Elovl6 gene in the intestine or/and further improved the ability to down-regulate expression of SCD1 gene in skeletal muscles of said mammal.

The invention is further illustrated in the following non-limiting examples and the figures.

### BRIEF DESCRIPTION OF THE DRAWINGS

FIG. 1. Total body fat content (dual energy x-ray absorptiometry), and chow consumption in germ-free (GF) and conventional (CONV) mice. Data from Bäckhed et al., 2004².

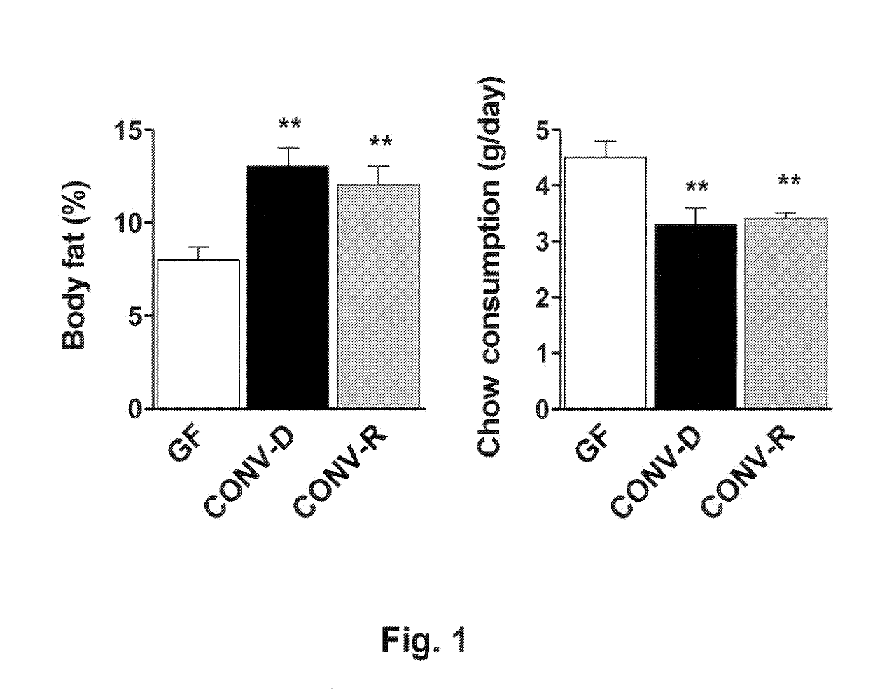

FIG. 2. Microbiota effects on triglyceride storage in adipocytes. Colonization suppresses intestinal FIAF expression, causing increased LPL activity, which increases adipocyte triglyceride storage.

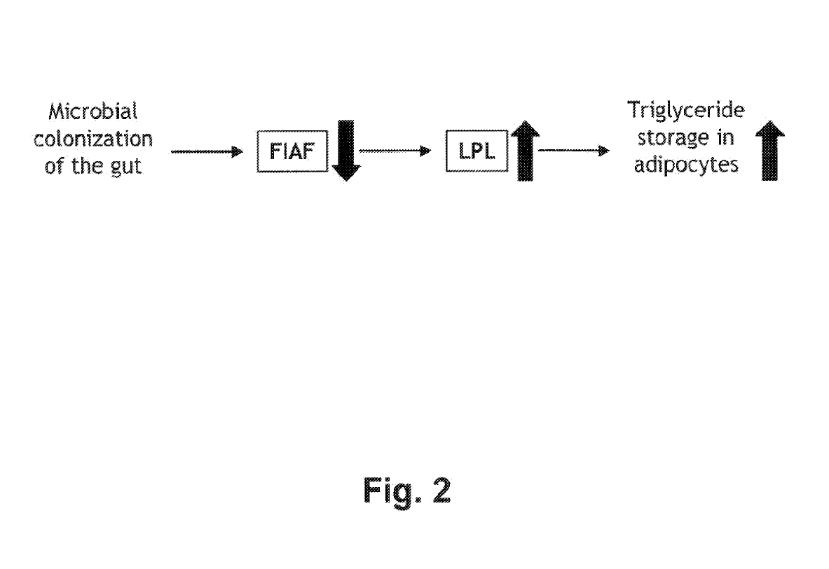

FIG. 3. Effects of Bifidobacterium animalis subsp. lactis BB-12 and Lactobacillus acidophilus LA-5 on triglyceride storage in adipocytes. Colonization with BB-12 or LA-5 improve microbiota suppressed intestinal FIAF expression, and decreases LPL activity and adipocyte triglyceride storage.

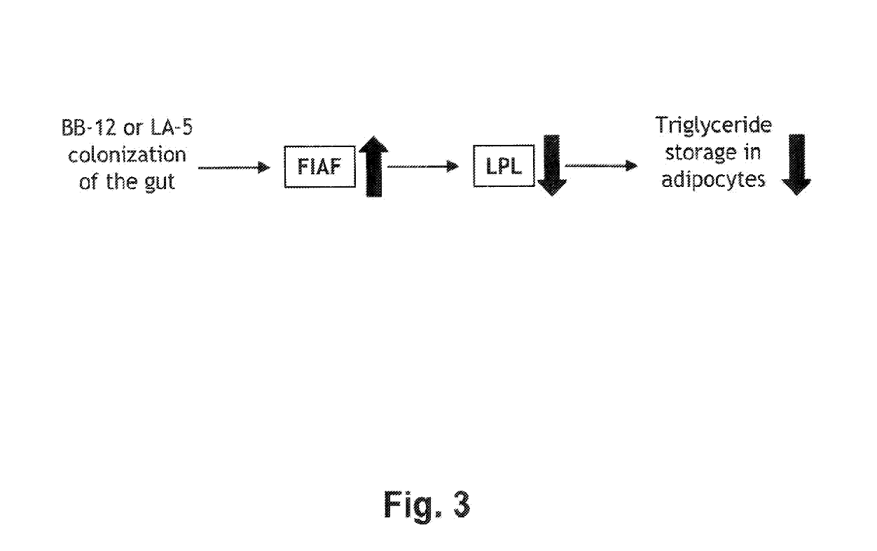

FIG. 4: Expression of ELOVL6 in porcine ileum. ELOVL6 expression was quantified by Q-PCR on RNA extracted from ileal samples. The average value of the control group (crtl) was set at 1.0 (7 pigs in group). Bb12: Bifidobacterium animalis subsp. lactis strain BB-12® (DSM15954) (7 pigs); LA-5: Lactobacillus acidophilus strain LA-5 (DSM13241) (6 pigs); CRL431: Lactobacillus paracasei subsp. paracasei strain CRL431 (ATCC 55544) (5 pigs).

FIG. 5. Expression of SCD-1 in skeletal muscle. SCD-1 expression was quantified by Q-PCR on RNA extracted from muscle samples. The average value of the control group (crtl) was set at 1.0 (7 pigs in group). Bb12: Bifidobacterium animalis subsp. lactis strain BB-12®(DSM15954) (7 pigs); LA-5: Lactobacillus acidophilus strain LA-5 (DSM13241) (6 pigs); CRL431: Lactobacillus paracasei subsp. paracasei strain CRL431 (ATCC 55544) (5 pigs).

FIG. 6. Standardized expression of ANGPTL4 in pig intestinal tissue determined by Q-PCR. ANGPTL4 expression was set to 1 in untreated control pigs (crtl) and fold-changes were determined relative to this in BB-12 (Bifidobacterium animalis subsp. lactis strain BB-12® (DSM15954)) and LA-5 (Lactobacillus acidophilus strain LA-5 (DSM13241)) treated pigs. Jejunum (SI25) A; Ileum (SI75) B; colon C. *P<0.05; **P<0.01; t-test.

### EXAMPLES

### Example 1

### Probiotic Strain Down-Regulate ELOVL6 Expression in the Ileum of Pigs

To investigate whether or not selected probiotic strains regulate ileal ELOVL6 expression in animals, young pigs were fed a standard diet including probiotic bacteria (i.e. Bifidobacterium animalis subsp. lactis strain BB-12® (DSM15954), Lactobacillus acidophilus strain LA-5 (DSM13241), and Lactobacillus paracasei subsp. paracasei strain CRL431, (ATCC 55544). Pigs fed with the same standard diet but not supplemented with probiotic bacteria served as control. Each group consisted of 8 piglets. At weaning at 4 weeks the animals were moved to pens where they were housed individually and assigned to the corresponding treatments for 14 days. Littermates were assigned to each of the treatments. The number of barrows and gilts in each treatment was the same. The pigs were fed twice daily, receiving an amount of feed corresponding to 4% of their body weight. The probiotics were given on top of the diet every morning.

Permission to carry out the experiment was granted from The Danish Plant Directorate and The Danish Ministry of Food, Agriculture and Fisheries.

After 14 days of treatment, the pigs were killed and tissues comprising 75% of the full length of the small intestine (i.e. the ileum or distal part of the small intestine) were sampled and snap-frozen in liquid nitrogen. Gene expression analysis on the distal ileum was performed by quantitative PCR analysis using primers specific for ELOVL6. The quantitative PCR (Q-PCR) analysis was performed essentially as described by Kubista et al.²².

Primer sequences were,

ELOVL6-F: 5′-CTA GCG AGT TTG CCA GCA C-3′
ELOVL6-R: 5′-TCC CTT GCT TCC CTC CTC-3′

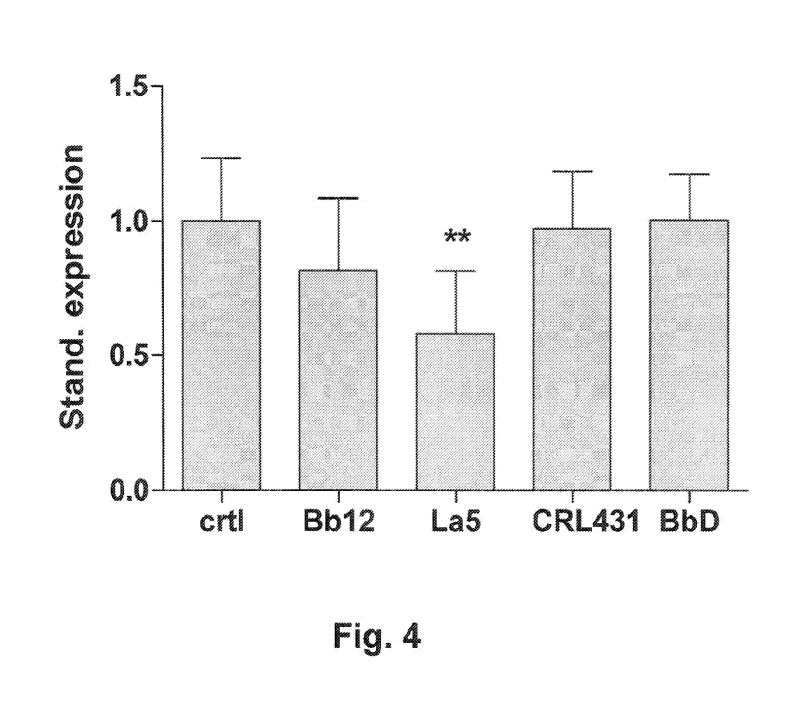

As indicated in FIG. 4, LA-5 significantly down-regulates ileal ELOVL6 expression (p=0.0057) while the two other strains had no significant effect on ELOVL6 expression.

### Example 2

### Probiotic Strain Down-Regulate SCD-1 Expression in the Skeletal Muscle of Pigs

To investigate whether or not selected probiotic strains regulate skeletal muscle SCD-1 expression in animals, young pigs were fed a standard diet including probiotic bacteria (i.e. Bifidobacterium animalis subsp. lactis strain BB-12® (DSM15954), Lactobacillus acidophilus strain LA-5 (DSM13241), and Lactobacillus paracasei subsp. paracasei strain CRL431, (ATCC 55544)) and otherwise treated as in example 1.

After 14 days of treatment, the pigs were killed and tissues comprising skeletal muscle were sampled and snap-frozen in liquid nitrogen. Gene expression analysis on the distal ileum was performed by quantitative PCR analysis using primers specific for SCD-1. The quantitative PCR analysis was performed essentially as described by Kubista et al.²².

Primer sequences were,

SCD1-F: 5′-GGG ATA CAG CTC CCC TCA TAG-3′
SCD1-R: 5′-AGT TCC GAT GTC TCA AAA TGC-3′

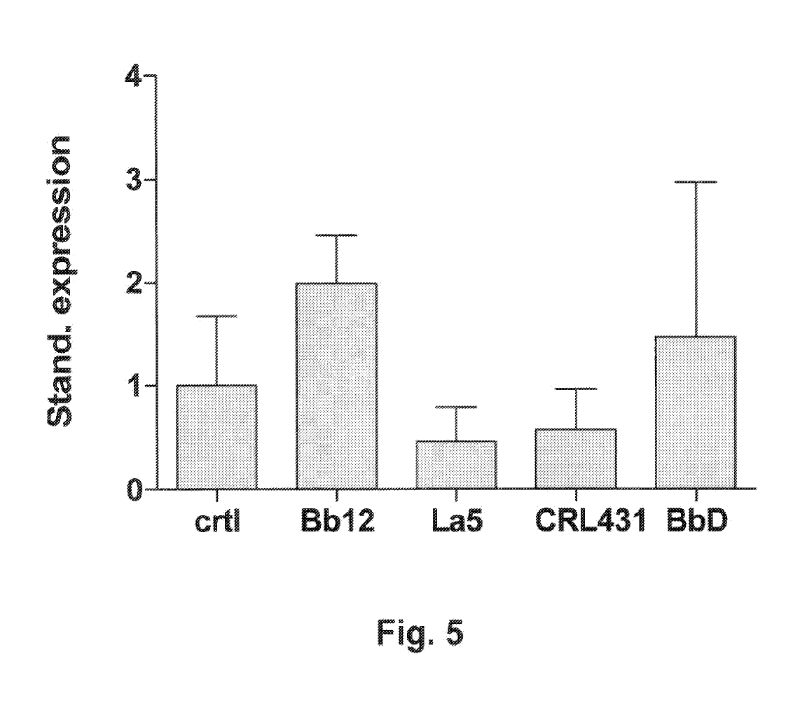

As indicated in FIG. 5, LA-5 down-regulates skeletal muscle SCD-1 expression by approximately half the level of the non-treated pigs. This is comparable to the down-regulation observed for CRL-431. In contrast, Bb-12 and BbD (inactivated, dead Bb-12) appears to up-regulate muscle SCD-1 by 100% (for Bb-12) compared to non-treated pigs.

### Example 3

### Probiotic Strains Up-Regulate ANGPTL4 Expression in the Jejunum, Ileum, and Colon of Pigs

To investigate whether or not selected probiotic strains regulate intestinal ANGPTL4 expression in animals, young pigs were fed a standard diet including probiotic bacteria, i.e. Bifidobacterium animalis subsp. lactis strain BB-12® (DSM15954) or Lactobacillus acidophilus strain LA-5 (DSM13241) and otherwise treated as in example 1.

After 14 days of treatment, the pigs were killed and tissues comprising 25% and 75% of the full length of the small intestine (i.e. the proximal and distal part of the small intestine) as well as the colon were sampled and snap-frozen in liquid nitrogen. Gene expression analysis on the intestinal samples was performed by quantitative PCR analysis using primers specific for GCG. The quantitative PCR analysis was performed essentially as described by Kubista et al.²².

Primer sequences were,

ANGPTL4-F: 5′-TCG ATG GCA GAT TCA GTC AC-3′
ANGPTL4-R: 5′-CCT GGG CCC TAC AGA AGT C-3′

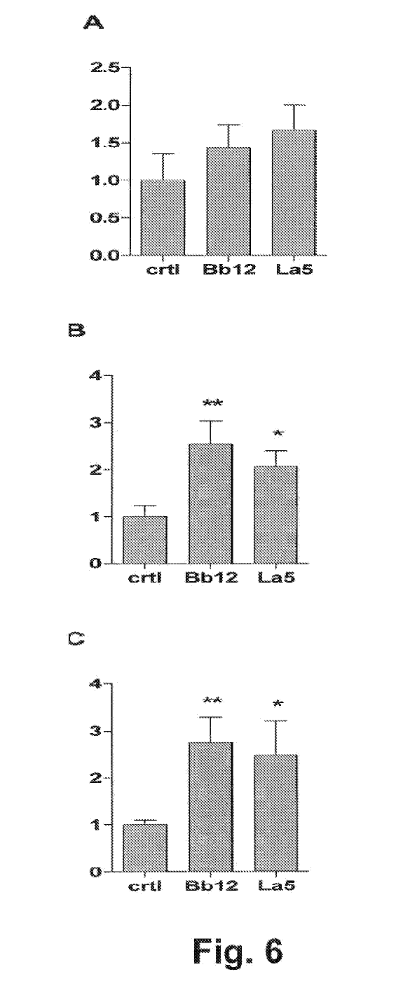

As indicated in FIG. 6, BB-12 and LA-5 significantly up-regulates ANGPTL4 expression in pig intestines compared to control fed animals.

### Example 4

### Effect of Probiotics on Body Weight or Insulin Resistance

To study the effects of the composition according to the invention, the following procedure may be used.

The study is a double blind, placebo controlled randomized study, done in parallel.

For the control group, a placebo dose is administered. For the active ingredient group, a dose of 10exp9-10exp10 of the probiotic bacterium is administered.

For measuring the effect on weight loss, the dose is given daily during 6 months, followed by measuring body weight. A loss of body weight will indicate the effectiveness of the composition according to the invention.

For measuring the effect on insulin resistance, the dose is given daily during 1 month, followed by measuring the levels of glycosylated hemoglobin, fasting glucose or insulin, and the HOMA index is calculated.

### REFERENCE LIST

1. Galgani, J. E., Uauy, R. D., Aguirre, C. A. & Diaz, E. O. Effect of the dietary fat quality on insulin sensitivity. Br. J Nutr 100, 471-479 (2008).
2. Backhed, F. et al. The gut microbiota as an environmental factor that regulates fat storage. Proc. Natl. Acad. Sci U. S. A 101, 15718-15723 (2004).
3. Riccardi, G., Giacco, R. & Rivellese, A. A. Dietary fat, insulin sensitivity and the metabolic syndrome. Clin Nutr 23, 447-456 (2004).
4. Marchand-Brustel, Y. et al. Fatty acid-induced insulin resistance: role of insulin receptor substrate 1 serine phosphorylation in the retroregulation of insulin signalling. Biochem Soc Trans. 2003 December; 31(Pt. 6):1152.-6. 31, 1152-1156 (2003).
5. Kruszynska, Y. T. et al. Fatty acid-induced insulin resistance: decreased muscle PI3K activation but unchanged Akt phosphorylation. J Clin Endocrinol Metab. 2002 January; 87. (1):226.-34. 87, 226-234 (2002).
6. Roche, H. M. Dietary lipids and gene expression. Biochem Soc Trans. 2004 December; 32. (Pt. 6):999.-1002. 32, 999-1002 (2004).
7. Boden, G. & Carnell, L. H. Nutritional effects of fat on carbohydrate metabolism. Best. Pract. Res Clin Endocrinol Metab. 2003 September; 17(3):399.-410. 17, 399-410 (2003).
8. Chakravarthy, M. V. et al. “New” hepatic fat activates PPARalpha to maintain glucose, lipid, and cholesterol homeostasis. Cell Metab 1, 309-322 (2005).
9. Ntambi, J. M. et al. Loss of stearoyl-CoA desaturase-1 function protects mice against adiposity. Proc. Natl. Acad Sci U. S. A 99, 11482-11486 (2002).
10. Ntambi, J. M. & Miyazaki, M. Recent insights into stearoyl-CoA desaturase-1. Curr. Opin. Lipidol. 14, 255-261 (2003).
11. Voshol, P. J., Rensen, P. C., van Dijk, K. W., Romijn, J. A. & Havekes, L. M. Effect of plasma triglyceride metabolism on lipid storage in adipose tissue: Studies using genetically engineered mouse models. Biochim. Biophys Acta (2009).
12. Li, C. Genetics and regulation of angiopoietin-like proteins 3 and 4. Curr. Opin. Lipidol. 17, 152-156 (2006).
13. Kersten, S. Regulation of lipid metabolism via angiopoietin-like proteins. Biochem Soc Trans. 33, 1059-1062 (2005).
14. Xu, A. et al. Angiopoietin-like protein 4 decreases blood glucose and improves glucose tolerance but induces hyperlipidemia and hepatic steatosis in mice. Proc. Natl. Acad Sci U. S. A 102, 6086-6091 (2005).
15. Oike, Y., Yasunaga, K. & Suda, T. Angiopoietin-related/angiopoietin-like proteins regulate angiogenesis. Int J. Hematol. 80, 21-28 (2004).
16. Matsuzaka, T. et al. Crucial role of a long-chain fatty acid elongase, Elovl6, in obesity-induced insulin resistance. Nat. Med 13, 1193-1202 (2007).
17. Matsuzaka, T. & Shimano, H. Elovl6: a new player in fatty acid metabolism and insulin sensitivity. J. Mol. Med (2009).
18. Hulver, M. W. et al. Elevated stearoyl-CoA desaturase-1 expression in skeletal muscle contributes to abnormal fatty acid partitioning in obese humans. Cell Metab 2, 251-261 (2005).
19. Backhed, F., Manchester, J. K., Semenkovich, C. F. & Gordon, J. I. Mechanisms underlying the resistance to diet-induced obesity in germ-free mice. Proc. Natl. Acad. Sci U. S. A 104, 979-984 (2007).
20. Neovius, M., Sundstrom, J. & Rasmussen, F. Combined effects of overweight and smoking in late adolescence on subsequent mortality: nationwide cohort study. BMJ 338, b496 (2009).
21. Samulin, J., Berget, I., Grindflek, E., Lien, S. r. & Sundvold, H. Changes in lipid metabolism associated gene transcripts during porcine adipogenesis. Comparative Biochemistry and Physiology Part B: Biochemistry and Molecular Biology In Press, Corrected Proof.
22. Kubista, M. et al. The real-time polymerase chain reaction. Mol. Aspects Med 27, 95-125 (2006).
23. FAO/WHO (2002) Joint FAO/WHO Expert Consultation on Evaluation of Health and Nutritional Properties of Probiotics in Food Including Powder Milk with Live Lactic Acid Bacteria, October 2001 (ftp://ftp.fao.org/docrep/fao/meeting/009/y6398e.pdf)

1. A composition comprising at least one probiotic Lactobacillius acidophilus strain and/or a fraction of said strain and/or metabolite of said strain for ameliorating or preventing diet-induced insulin resistance, said composition is characterized by up-regulating expression of the ANGPTL4 gene encoding for FIAF in the intestine, down-regulating expression of the Elovl6 gene in the intestine as well as down-regulating expression of the SCD1 gene in skeletal muscles of a mammal, and wherein the probiotic strain is selected from the group of strains consisting of Lactobacillus acidophilus strain LA-5 (DSM13241), and a mutant strain thereof, wherein the mutant strain is obtained by using DSM13241, and wherein the mutant has retained or further improved the ability to up-regulate expression of the ANGPTL4 gene or/and further improved the ability to down-regulate expression of the Elovl6 gene in the intestine or/and further improved the ability to down-regulate expression of SCD1 gene in skeletal muscles of said mammal.

2. The composition according to claim 1 for ameliorating, treating or preventing a disease or condition selected from the group of obesity and obesity-related diseases consisting of obesity-induced insulin resistance, cardiovascular diseases (e.g. atherosclerosis, hypertension, stroke, congestive heart failure, Angina pectoris), type 1 diabetes mellitus, type 2 diabetes mellitus, metabolic syndrome, leptin resistance,
obesity-related hypoventilation, back and joint problems, non-alcoholic fatty liver disease, gastroesophageal reflux disease, reduced fertility, hypothyroidism, dyslipidemia, hyperinsulinemia, cholecystitis, cholelithiasis, osteoarthritis, gout, sleep apnea and other respiratory problems, polycystic ovary syndrome (PCOS), pregnancy complications, psychological disorders, uric acid nephrolithiasis (kidney stones), stress urinary incontinence and certain cancers (e.g. cancer of the kidney, endometrium, breast, colon and rectum, esophagus, prostate and gall bladder).

3. A cosmetic method for reducing body weight in a non-obese, non-overweight subject having a Body Mass Index (BMI) less than 25, said method comprise providing a composition comprising at least one strain of a probiotic Lactobacillius acidophilus strain and/or a fraction of said strain and/or metabolite of said strain, wherein said composition is characterized by up-regulating expression of the ANGPTL4 gene encoding for FIAF in the intestine, down-regulating expression of the Elovl6 gene in the intestine as well as down-regulating expression of the SCD1 gene in skeletal muscles of a mammal, and wherein the probiotic strain is selected from the group of strains consisting of Lactobacillus acidophilus strain LA-5 (DSM13241), and a mutant strain thereof, wherein the mutant strain is obtained by using DSM13241, and wherein the mutant has retained or further improved the ability to up-regulate expression of the ANGPTL4 gene or/and further improved the ability to down-regulate expression of the Elovl6 gene in the intestine or/and further improved the ability to down-regulate expression of SCD1 gene in skeletal muscles of said mammal.

4. A cosmetic method for reducing body weight in a non-obese subject, said method comprise providing a composition comprising at least one strain of a probiotic Lactobacillius acidophilus strain and/or a fraction of said strain and/or metabolite of said strain, wherein said composition is characterized by up-regulating expression of the ANGPTL4 gene encoding for FIAF in the intestine, down-regulating expression of the Elovl6 gene in the intestine as well as down-regulating expression of the SCD1 gene in skeletal muscles of a mammal, and wherein the probiotic strain is selected from the group of strains consisting of Lactobacillus acidophilus strain LA-5 (DSM13241), and a mutant strain thereof, wherein the mutant strain is obtained by using DSM13241, and wherein the mutant has retained or further improved the ability to up-regulate expression of the ANGPTL4 gene or/and further improved the ability to down-regulate expression of the Elovl6 gene in the intestine or/and further improved the ability to down-regulate expression of SCD1 gene in skeletal muscles of said mammal.

5. The composition according to claim 1, wherein the at least one strain and/or a fraction and/or metabolite is used for the preparation of a food or feed intended to ameliorate or prevent diet-induced insulin resistance of a mammal.

6. The composition according to claim 1, wherein the at least one strain and/or a fraction and/or metabolite is combined with at least one prebiotic.

7. A composition according to claim 6, wherein the at least one strain and/or a fraction and/or metabolite is combined with at least one prebiotic, wherein the at least one prebiotic is selected from the group consisting of: inulin, a transgalacto-oligosaccharide, palantinoseoligosaccharide, soybean oligosaccharide, gentiooligosaccharide, oxylooligomers, nondegradable starch, lactosaccharose; lactulose, lactitol, maltitol, FOS (fructo-oligosaccharides), GOS (galacto-oligosaccharides), and polydextrose.

8. A use of at least one strain one probiotic Lactobacillius acidophilus strain and/or a fraction of said strain and/or metabolite of said strain for the preparation of a medicament for administration to a mammal for treating, ameliorating or preventing diet-induced insulin resistance, said composition is characterized by up-regulating expression of the ANGPTL4 gene encoding for FIAF in the intestine, down-regulating expression of the Elovl6 gene in the intestine as well as down-regulating expression of the SCD1 gene in skeletal muscles of a mammal, and wherein the probiotic strain is selected from the group of strains consisting of Lactobacillus acidophilus strain LA-5 (DSM13241), and a mutant strain thereof, wherein the mutant strain is obtained by using DSM13241, and wherein the mutant has retained or further improved the ability to up-regulate expression of the ANGPTL4 gene or/and further improved the ability to down-regulate expression of the Elovl6 gene in the intestine or/and further improved the ability to down-regulate expression of SCD1 gene in skeletal muscles of said mammal.
